(19) United States
(12) Patent Application Publication
(10) Pub. No.: US 2017/0007200 A1
(43) Pub. Date: Jan. 12, 2017

(54) Ultrasound Guidance System Including Tagged Probe Assembly

(71) Applicant: Soma Research, LLC, Greenville, SC (US)

(72) Inventors: M. Dexter Hagy, Greenville, SC (US); Stephen Ridley, Columbia, SC (US); Michael R. LaBree, Denver, CO (US); Lawrence Busse, Fort Mitchell, KY (US)

(21) Appl. No.: 15/270,596

(22) Filed: Sep. 20, 2016

Related U.S. Application Data
(63) Continuation of application No. 13/835,034, filed on Mar. 15, 2013.

(51) Int. Cl. A61B 8/08; A61B 8/00; A61M 5/32; A61B 17/34; A61B 90/98; A61B 5/06; A61B 10/02

(52) U.S. Cl. CPC A61B 8/0841; A61B 5/062; A61B 5/066; A61B 8/4254; A61B 10/0233; A61B 17/3403; A61B 90/98; A61M 5/3287; A61B 2017/3413; A61B 2017/347

(57) ABSTRACT

Ultrasound-based systems are described for use in guiding subdermal probes during medical procedures. The systems include an ultrasound system in conjunction with a probe detection system. The probe detection system can be used to generate a virtual image of a probe in a subdermal environment such that the virtual image is highly correlated with the actual probe location in the subdermal environment. The probe used in the system can include a tag that can provide information concerning the probe characteristics to the probe detection system.

### CROSS REFERENCE TO RELATED APPLICATION

This application is a continuation application and claims priority to U.S. patent application Ser. No. 13/835,034 having a filing date of Mar. 15, 2013, which is incorporated herein by reference in its entirety.

### BACKGROUND OF THE INVENTION

Medical probe devices are utilized for many purposes, chief of which include catheterization, centesis, and biopsy procedures. Subdermal placement of probes using these devices is often performed with techniques that rely on ascertaining the correct locations of palpable or visible structures. This is neither a simple nor a risk-free procedure. For instance, proper insertion and placement of a probe depends on correct localization of anatomical landmarks, proper positioning of the patient in relation to the care provider, and awareness of both the target's depth and angle from the point of probe insertion. Risks of unsuccessful placement of a probe can range from minor complications, such as patient anxiety and discomfort due to repetition of the procedure following incorrect initial placement, to severe complications, such as pneumothorax, arterial or venous laceration, or delivery delay of life-saving fluids or medications in an emergency situation.

To improve proper placement of subdermal probes, devices such as ultrasound transducers are often utilized. Ultrasound guided techniques often utilize two people, an ultrasound operator who locates the internal target and keeps an image of the target centrally located on a monitor, and a care provider who attempts to guide the probe to the target based upon the sonogram. Such techniques are very difficult perceptually as the probe itself is virtually invisible on the sonogram, but have greatly improved the ability to properly place a subdermal probe.

Computer aided probe placement has been developed, in which probe detection and spatial analysis is utilized to provide additional information to the medical staff with regard to where the probe is located in relation to the anatomical features that are visibly detectable on the sonogram. Visualization systems have been described previously, for instance in U.S. Pat. Nos. 7,244,234 and 8,152,724 to Ridley, et al., and in U.S. Patent Application Publication Nos. 2012/0157855, 2012/0157849, 2011/0087106, and 2011/0087105 to Ridley, et al., all of which are incorporated herein by reference thereto.

Such methods require high correlation between the analytical system and the ultrasound system, as even a slight error in the analytical system specifications (e.g., probe characteristics, probe path, etc.) can lead to a lack of correlation between where the system reports the location of the probe to be and the actual location of the probe. Such a lack of correlation can lead to severe consequences, such as insertion of the probe in the wrong blood vessel.

What are needed in the art are improved probe devices and methods for using the devices. For instance, what are needed in the art are probe devices and systems that can guide a probe to a subdermal target with high accuracy.

### SUMMARY OF THE INVENTION

According to one embodiment, disclosed herein is a probe assembly that includes a probe (e.g., a needle) that has a first and second end, the first end of the probe including a probe tip for subdermal insertion. In addition to the probe, the probe assembly includes a target that is detectable by a detector. The probe assembly also includes a tag, the tag including information that can be used to identify the geometry of the subdermal probe.

According to another embodiment, an ultrasound system is disclosed. The system can include a monitor and a housing for an ultrasound transducer. The system can also include at least one detector (which differs from the ultrasound transducer), and a probe assembly that includes a probe that is configured for being guided to a subdermal location. The probe assembly includes a tag that includes information regarding the geometry of the probe. The probe assembly also includes a target that is detectable by a detector. The system also includes a probe guide that is attachable to the transducer housing. Upon attachment of the probe guide to the housing, the probe guide defines a barrier between a probe passing through the probe guide and the housing, such that contact is precluded between the probe and the housing. The system also includes a processor that is in communication with the detector, the probe assembly, the monitor, and the ultrasound transducer. The processor can be configured for creating and displaying a real time image of a virtual probe on the monitor. More specifically, the processor can be programmed to analyze data from the detector and the tag to calculate a relative position of the probe in relation to a reference point, the processor can then communicate the relative position of the probe to the monitor.

A method for guiding a subdermal probe to a target is also described. For example, a method can include guiding a probe through a probe guide to a subdermal location. The probe can be a component of a probe assembly that can also include tag that includes information with regard to the geometry of the probe. The probe assembly can also include a target for a detector. An ultrasound transducer is used during the method to form a sonogram of the subdermal location on a monitor. The method can also include detecting the motion of the probe in the probe guide by use of a detector, creating a data stream in response to the detected motion, and utilizing a processor that is in communication with the detector, the probe assembly, the monitor, and the ultrasound transducer to process information contained in the data stream and information of the tag to form a real time image of a virtual probe on the monitor. More specifically, the processor can be programmed to calculate a relative position of the probe in relation to a reference point, and can be capable of communicating the relative position to the monitor such that the relative position can be displayed in conjunction with the sonogram on the monitor as the real time image of the virtual probe.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 illustrates one embodiment of an ultrasound system as disclosed herein.

FIG. 2A illustrates an ultrasound device including a series of Hall effect sensors along a length of the ultrasound device.

FIG. 2B illustrates one embodiment of an array of Hall effect sensors as may be utilized in disclosed ultrasound devices.

FIG. 2C illustrates another embodiment of an array of Hall effect sensors as may be utilized in disclosed ultrasound devices.

FIG. 3 illustrates one embodiment of an ultrasound transducer housing as disclosed herein.

FIG. 4 illustrates a bottom view of the ultrasound transducer housing of FIG. 3.

FIG. 5 illustrates one embodiment of an ultrasound system during use.

FIG. 6 illustrates a lower section of a sterilizable shield that can be utilized in conjunction with an ultrasound transducer housing as is illustrated in FIG. 3.

FIG. 7 illustrates the upper section of the sterilizable shield, the lower section of which is illustrated in FIG. 6.

FIG. 8 illustrates another embodiment of an ultrasound transducer housing.

FIG. 9A is an exploded view of a system that can incorporate the ultrasound transducer housing of FIG. 8.

FIG. 9B illustrates the system of FIG. 9A following assembly.

Repeat use of reference characters in the present specification and drawings is intended to represent the same or analogous features of elements of the disclosed subject matter. Other objects, features and aspects of the subject matter are disclosed in or are obvious from the following detailed description.

### DETAILED DESCRIPTION OF PREFERRED EMBODIMENTS

Reference will now be made in detail to various embodiments of the disclosed subject matter, one or more examples of which are set forth below. Each embodiment is provided by way of explanation of the subject matter, not limitation of the subject matter. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment. Thus, it is intended that the present disclosure cover such modifications and variations as come within the scope of the appended claims and their equivalents.

In general, disclosed herein are systems and methods for use in forming a virtual image of a probe in conjunction with a sonogram during a medical procedure. More specifically, disclosed herein are systems that can include an ultrasound system in conjunction with a probe detection system. The probe detection system can include a probe assembly and can be used to generate a virtual image of a probe in a subdermal environment such that the virtual image is highly correlated with the actual probe location in the subdermal environment. To help achieve this high correlation, the probe assembly used in the system can include a tag that can provide to the system information concerning the probe characteristics (e.g., geometric characteristics). The probe assembly can also include a target that can be detected by a detector. The detection of the target can provide information to the system concerning the motion of the probe. As utilized herein, the term “probe” generally refers to a device that can be guided to a subdermal location, for instance for delivery of a therapeutic, e.g., a compound or a treatment, to the location; for removal of material from the location; and so forth. For example, the term “probe” can refer to a needle, a tube, a biopsy device, or any other item that can be guided to a subdermal location. In general, a probe can be guided by and used in conjunction with an ultrasound device as described herein. A probe assembly can include the probe in conjunction with one or more additional components including the tag and target as described herein as well as any standard components as are known in the art such as, without limitation, a syringe, a catheter, a needle hub, a stylet, and so forth.

The probe detection system can include a detector that can recognize the target and that can be placed in direct or indirect communication with a processor. The processor utilizes information received from the detector and also from the tag of the probe assembly to identify the location of the probe tip in a subdermal location. The processor can also be in communication with a monitor and can create an image of a virtual probe on the monitor, generally in conjunction with the sonogram. Beneficially, the system can accurately correlate the image of the virtual probe, and particularly the probe tip, with the actual location of the subdermal probe.

During a medical procedure, the probe can be guided through a probe guide and the probe tip can approach a subdermal site that can be visualized on the scanned plane of a sonogram. The probe guide can be designed such that the probe tip can travel on a path that defines a known correlation with sound waves emitted by the ultrasound transducer, e.g., coincident in the scanned plane, parallel to the scanned plane, or intersecting the scanned plane. When utilizing the ultrasound device, the path of the probe to the subdermal site can be known: the probe can advance toward the subdermal site on a straight line and at a predetermined angular relationship to the emitted sonic waves. The probe can advance from the probe guide opening to the subdermal site that is imaged by the ultrasound. Thus, the path of the probe and the scanned plane of the sonogram image can both be defined by the orientation of the ultrasound transducer and can be coordinated on the subdermal site. In order to strike the site, the probe tip can be guided along this known path the desired distance. Beneficially, the system can be conveniently utilized by a single operator who can insert the probe and also control the ultrasound transducer so as to see the sonogram and the virtual image of the probe overlaid on the sonogram in real time during the procedure.

The probe detection system can include a detector that can register the location of a target on the probe assembly. This information can be electronically communicated to a processor and processed with the data provided from the tag of the probe assembly and any other desired input data and displayed as a real time image of a virtual probe in conjunction with a sonogram, i.e., the two images, the virtual image developed from the data obtained by the probe detection system, and the sonogram developed from the data obtained from the ultrasound transducer, can be displayed on the same monitor. Because the virtual probe location is well correlated with the actual probe location, the location of the probe tip in relation to the subdermal site and the striking of the subdermal site by the probe tip can be seen in real time by an operator watching the virtual probe on the monitor during the procedure.

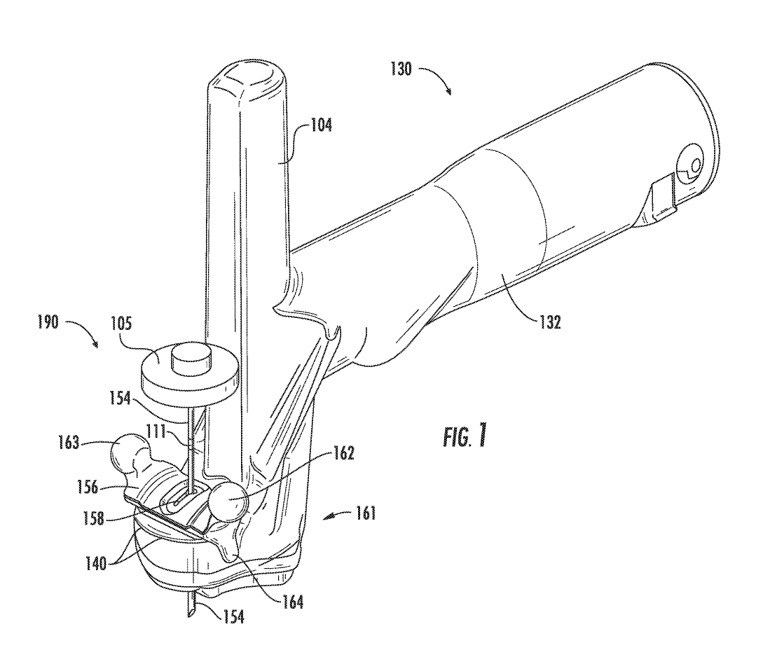

One embodiment of an ultrasound device 130 is illustrated in FIG. 1. As can be seen, the device 130 includes a handle 132 and a base 161. During use, the base 161 can be pressed against the skin of a subject, and an ultrasound transducer held in the base 161 can transmit and receive ultrasonic signals according to known methodology. The device 130 also includes a clamp 156 that can be attached to the upper surface 140 of the base 161. The clamp 156 includes features 162, 163 that can allow a user to pivot the clamp 156 about a pivot point 164. As the clamp pivots, an aperture 158 slides across a probe 154 guided by use of the device to clamp and hold the probe 154 at a desired location.

The probe 154 is a component of the probe assembly 109. In the embodiment of FIG. 1, the probe assembly includes a target 105 and a tag 111. The tag 111 can include information about the probe and the probe assembly including, without limitation, the probe type (e.g., needle, biopsy device, etc.) as well as the probe geometry such as probe gauge, length, cross section, etc. The information from the tag can be utilized to accurately determine a characteristic distance of the probe assembly, for instance the distance from the center of the target 105 to the tip of the probe 154, which can then be used to accurately correlate the location of the probe tip as determined by the probe detection system with the actual location of the probe tip in the subdermal environment.

In one embodiment, the identification method can determine an identifying reference (e.g., a single number identifying the probe) that is carried by the tag 111, for instance in the form of an information chip. This reference can then be transmitted to the processor that can be preprogrammed to recognize the code and access the preprogrammed information needed for identifying the characteristics of the probe 154. Alternatively, the tag can be designed to directly carry the desired information (e.g., geometric information) that describes the probe 154.

The tag 111 can be located at any convenient point on the probe assembly, and is not limited to location on the probe 154 as illustrated in FIG. 1. For instance, the tag may be located on the needle hub, or may be a component of the target 105, as discussed further herein. In addition, the probe assembly may be any assembly as is generally known in the art. For instance, the probe assembly can include a stylet, a syringe, a multi-component hub, a butterfly grip, and so forth, and the tag may be located on or in any component of the probe assembly. For instance, in one embodiment the tag may be located on a stylet or a stylet hub.

The tag 111 can use any of a variety of technologies to provide information to a processor of the ultrasound system. In one embodiment, the tag 111 can be a radio-frequency identification (RFID) tag. An RFID tag can be a passive type or an active type of RFID tag as is known in the art. By way of example, RFID tags as described in U.S. Pat. No. 8,174,368 to Usami, U.S. Pat. No. 8,035,522 to Oroku, et al., U.S. Pat. No. 8,325,047 to Marur, et at, and U.S. Pat. No. 7,876,228 to Kroll, et al., all of which are incorporated herein by reference, can be utilized in the probe detection system.

In one embodiment, such as illustrated in FIG. 1, in which the tag 111 is located on or in the probe 154, the tag 111 can be a component of a passive RFID device. A passive RFID device has no on-board battery and transmits its information from the RFID tag 111 in response to the temporary delivery of power from an RFID transceiver, which is part of a tag sensor (not illustrated in FIG. 1) that can be located, for example, in the base 161, the post 104, or the handle 132 of device 130. The RFID transceiver can use an antenna to transmit power to the tag 111 via low frequency RF signals. The RFID tag 111 then uses the received power to transmit the information contained in the RFID tag 111 back to the transceiver.

Low-frequency RFID signals are generally employed, e.g. signals operating at or below about 125 kHz. By using low-frequency signals, the signals can properly propagate. Optimal transmission power values to be used can depend upon the size, shape and orientation of the antenna of the transceiver, its proximity during operation to the tag 111, as well as the characteristics of the RFID tag 111. Routine experimentation may be performed to identify optimal power transmission levels based upon these parameters. Routine experimentation may also be employed to determine optimal parameters for the size, shape, position and orientation of the antenna of the transceiver.

Functional components for passive RFID tags can generally include an RF rectifier (which is used as a power supply), an ID circuit (which stores the information of the RFID tag), control logic and an on-chip antenna. The ID circuit may be a read-only memory (ROM) circuit. The sensor can include an antenna, a transceiver and control logic for controlling transmission and reception of signals from the RFID tag as well as for transmitting the signals from the RFID tag to a processor. Briefly, in the passive RFID implementation, the control logic of the sensor controls the transceiver to deliver alternating current (AC) power to an antenna for transmission via an RF link to the RFID tag. AC power received by the antenna is rectified by the RF rectifier, which is then routed to the control logic of the RFID tag, which uses the power to access the ID ROM to readout the RFID tag and to transmit the RFID tag information via the antenna to the sensor over the RF link. As noted above, low frequencies are preferably used. The information transmitted by the RFID tag is received by the antenna of the sensor and decoded by its transceiver. The control logic of the sensor uses the RFID tag in accordance with the techniques described above to identify the particular probe that is incorporated in the probe assembly.

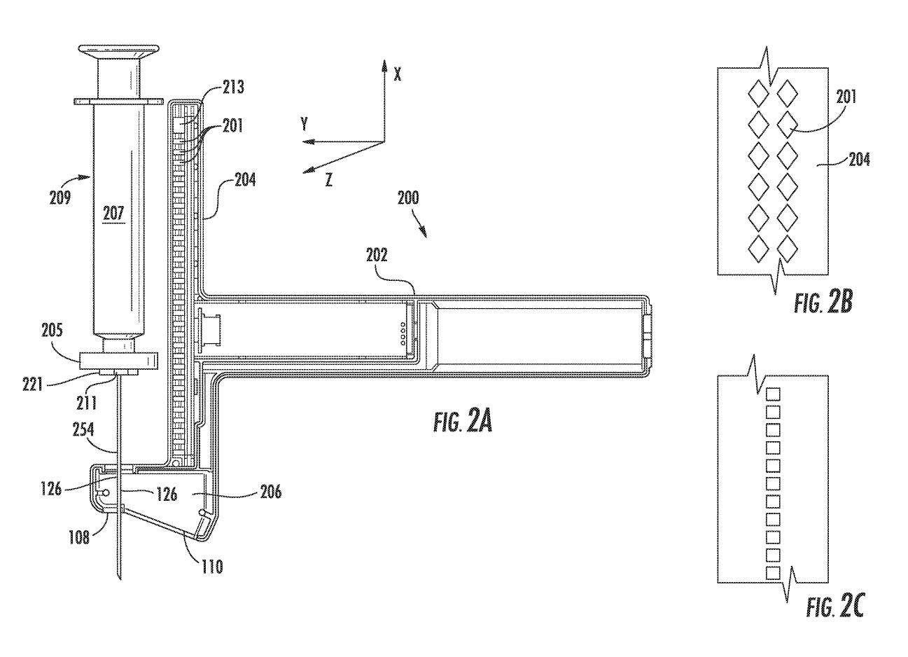

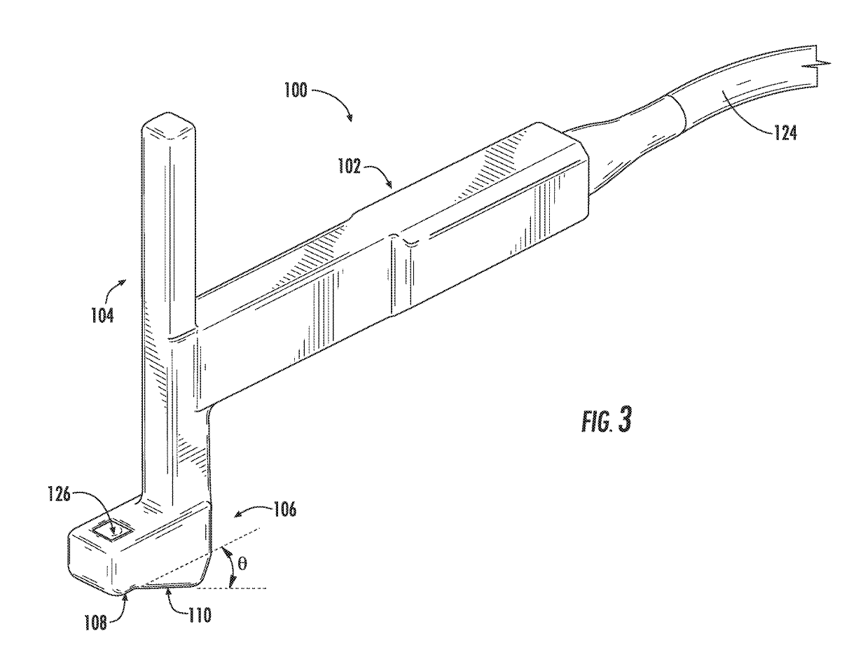

As noted, the RFID device can be an active or a passive device. In general, in those embodiments in which the RFID device is an active device, the RFID tag can be located on the probe assembly in a location that is conducive to an active tag. For instance, and as illustrated in FIG. 2A, the RFID tag 211 can be located on or in a support 221 that supports the target 205 and the sensor 213 can be located in the post 204 of the device 200. For example, the support 221 can be a portion of the needle hub, a stylet hub, a syringe, or so forth.

Briefly, an active RFID device can include an on-board battery, an ID circuit, control logic and an antenna. Whereas the antenna of the passive RFID device must be capable of receiving power from the transceiver as well as transmitting RFID signals, with the active device, power is instead provided by the on-board battery and hence the antenna is used only to transmit data. Accordingly, the antenna of the active device may differ in size and configuration from the antenna of the passive device. The tag sensor 213 of the active device can include an antenna, a transceiver and control logic for controlling reception of signals from the RFID tag 211 and communicating information to the processor. Whereas an antenna for use with a passive RFID device should be capable of transmitting power to the RFID tag, antenna of the active device need only receive signals from the RFID tag. The transceiver and control logic of the active device may differ from corresponding components of the passive device, as is known.

In the embodiment illustrated in FIG. 2A, the probe assembly 209 can include a one-piece support 221 capable of holding the RFID tag 211. The one-piece support 221 may include a thermoplastic substrate onto which the RFID tag 211 is located, or may include a thermoplastic article having a cavity into which the RFID tag 211 is located. In each instance, a thermoplastic seal material can be overmolded around the support 221 to form a resulting barrier structure that can provide increased break strength to the RFID tag 211 via the characteristics of the overmolded seal material while also providing enhanced thermal resistance since the components of the RFID tag 211 can be encased with the seal material, which acts as a barrier layer during sterilization conditions, thereby giving the RFID tag 211 enhanced thermal resistance. In addition, since an overmolding step is used, there are two layers of thermoplastic material, which increases the break strength of the RFID tag 211. Depending on the type of overmolding step or steps performed, seams can also be substantially reduced or even eliminated, thereby further increasing the break strength and/or thermal resistance of the RFID tag 211.

The overmolded seal layer can encompass or substantially encompass the support 221 and RFID tag 211. By encompassing the support 221 and tag 211, the material characteristics of the seal material, as it relates to break strength and moisture barrier, can provide enhanced break strength and/or thermal resistance to the RFID tag 211. If a stronger RFID article is desired, a stronger support material and/or sealing material may be used. If greater temperature resistance and/or moisture barrier are desired, a plastic material having high heat deformation temperature and/or moisture prevention may be used.

The tag is not limited to an RFID tag, and other types of tags may alternatively be utilized. For example, in one embodiment the tag may be an optical tag, and can utilize optical methods including, without limitation, QR- or Bar-code, color coding, etc. By way of example, a bar code can be printed on the target, the needle hub, the syringe, or any other suitable component of the probe assembly. As the needle assembly passes an optical sensor located, for example, on the post 204, it can be read by the sensor (with rotation of the assembly, if necessary) and the information can be sent to the processor.

In conjunction with the tag of the probe assembly, the probe detection system also includes a target on the probe assembly and a detector located at a distance from the probe assembly to detect the presence and/or motion of the probe. In general, any suitable detector can be utilized in the detection system for detecting the probe. For instance, a detector can utilize infrared (IR), ultrasound, optical, laser, magnetic, or other detection mechanisms. In addition, the location of the target and the detector is not critical to a device, save that it is capable of detecting the target that is associated with the probe assembly. In addition, the target can be any suitable item. It can be all or a portion of the probe itself, or can be directly or indirectly attached to the probe as a component of the probe assembly. For instance, it can be on or near a needle hub, a syringe, or any other component of the probe assembly.

FIG. 2A illustrates one embodiment of a magnetic based ultrasound device and probe detection system. As can be seen, the ultrasound device 200 includes a handle 202, a post 204, and a base 206. The base 206 can define a probe guide opening 126 therethrough, and an ultrasound transducer that transmits and receives ultrasonic waves can be located in base 206. The probe assembly 209 includes a syringe 207, magnetic target 205, tag 211, and probe 254. As can be seen, in this embodiment, the tag 211 is located on the support 221 beneath the target 205. Of course, in other embodiments, the probe assembly may include other components, as is known in the art.

In one embodiment, the tag can be a component of the magnetic target 205. For example, probe identification can be carried out by use of differences in magnetic targets, as variations in the magnetic target will vary the magnetic field associated with the target. For example, variation in strength of the magnetic field can be utilized to identify the characteristics (size, type, etc.) of the probe 254. Other variations in magnetic targets that can be used for probe identification can include, without limitation, variations in size and shape (e.g., width) of a magnetic target; variations in the number and relative locations of magnets used to form a magnetic target; the orientation of multiple magnets used to form a magnetic target (e.g., the arrangement of the north and south poles of the multiple magnets of the target); variation in shape of the magnetic target; and so forth. In such a case, the detector used to detect the target could also detect the information carried by the tag, e.g., the detector would gather data that would convey not only information with regard to the presence and/or motion of a probe in the probe guide, but also information concerning the geometry and other information about of the probe.

Referring again to FIG. 2A, the ultrasound device 200 can include a series of sensors 201 that form a detector along a length of post 204. Sensors can be sensitive to the presence of the magnetic target 205. In the magnetic based detection system, sensors 201 can be Hall effect sensors that are sensitive to a magnetic field and target 205 can include one or more magnets. One exemplary embodiment of a magnetic based detection system as may be incorporated in disclosed devices is describe in U.S. Pat. No. 6,690,159 to Burreson, et al. and U.S. Patent Application Publication No. 2013/0041254 to Hagy, et al., which are incorporated herein by reference.

The sensors 201 can be arranged in one or more rows extending lengthwise along the post 204, which is the direction along which the probe will move during insertion, herein defined as the X direction, as shown in FIG. 2A. As is known, the presence of a magnetic field can induce a voltage in a Hall effect sensor that is proportional to the size of the magnetic field. The voltage of each sensor 201 can be electronically scanned and processed to determine the location of the target 205 relative to the sensing array (i.e., the detector). Processing can include grouping the sensors 201 and providing their outputs to a series of multiplexers which, in turn, are connected to a processor including software for analyzing the outputs and determining the location of the target 205 with regard to the entire sensor array. As the distance from the target 205 to the tip of the probe 254 can be provided to the processor by use of the tag 211 of the probe assembly 209, the processor can likewise compute the location of the tip of probe 254.

The processing of the sensor outputs can include determining which sensor 201 has the highest (or lowest, depending upon the magnetic field orientation) voltage output in a recognized grouping, corresponding to the location of the magnetic target 205. In one embodiment, a processor can analyze the output of the sensor having the highest voltage output and a predetermined number of sensor(s) to each side. The analog outputs of the sensors can be converted to digital output according to known methodology that can then be evaluated to determine the target location.

Other methods can also be used to determine a set of sensors to evaluate for position. One such method is correlation. In this method, a vector of values corresponding to the desired signal can be mathematically correlated against the vector signal set from scanned sensors 201. A peak in the correlation signal can indicate the center of the desired sensor set to evaluate.

Of course, the detection system need not utilize the peak signal and adjacent Hall effect sensors, but instead or in addition, sensors can evaluate the zero crossing signal that can result from using combinations of north and south magnets.

In the embodiment of FIG. 2A, the probe assembly 209 includes the magnetic target 205 mounted on the support 221 at the base of syringe 207 and in conjunction with probe 254. This particular arrangement is not a requirement of disclosed systems, however, and more details concerning suitable magnet assemblies are described in U.S. Pat. No. 5,285,154 to Burreson, et al. and U.S. Pat. No. 5,351,004 to Daniels, et al., both of which are incorporated herein by reference.

The magnetic material of target 205 can be any suitable material that provides a sufficiently high magnetic field strength to be detectable over the distance between the target 205 and the sensors 201. A non-limiting list of suitable materials can include, without limitation, samarium cobalt, neodymium, or iron boron.

In one embodiment, a row of sensors 201, e.g., Hall effect transducers, can be placed side by side in a single row in the X direction along the post 204, as illustrated in FIG. 2C. However, the distance between adjacent sensors can be affected by connection pins, casings, housings in which they are mounted, etc. For example, a small sensing component can be mounted in conjunction with pins or contacts that project from a housing for connection to a supply voltage, ground and output, respectively. Thus, even if housings are placed end to end with their pins projecting in the same or alternate directions, there will be a certain center to center distance between adjacent sensors. This distance can be reduced by providing an array of sensors that are canted at an angle to the sensing or X direction, and are provided in two rows with the sensors staggered relative to each other, as illustrated in FIG. 2B. This can decrease the center to center distance between adjacent sensing components for increased accuracy of a detector. Of course, other arrangements of the individual sensors 201 forming an array along post 204 are likewise encompassed in the present disclosure.

The Hall effect sensors can operate at a typical supply voltage of about 5 volts. According to one embodiment, all of the sensors 201 can be mounted on a single printed circuit board. The printed circuit board also can include multiplexers for scanning of the outputs of the sensors. For example, in the case of 64 sensors, eight eight-port multiplexers can be used and coupled to a processor. A ninth multiplexer can be used to take the output of the eight multiplexers to one output for an analog-to-digital converter.

Each multiplexer can receive the outputs from eight of the Hall effect sensors and can provide a selected output on a line to a processor. The processor can include an analog-to-digital converter that, in combination with the multiplexers, scans the outputs of the sensors and converts the signals to digital form. The processor can also store an algorithm by which the Hall array outputs (i.e., the location of the target) and the information from the tag 211 can be processed to determine the location of the tip of the probe relative to the sensor having the reading that locates that particular sensor closest to the center of the magnetic target 205, for example, the sensor closest to the center of magnetic target can be the sensor obtaining the highest voltage output reading.

Signals from the target sensors 201 and tag sensor 213 can create a data stream which can be sent to a processor. A processor can be internal or external to an ultrasound device 200. For example, data from sensors 201, 213 can be directly or indirectly sent to a standard laptop or desktop computer processor or part of a self-contained ultrasound device as is known in the art. A processor can be loaded with suitable recognition and analysis software and can receive and analyze the stream of data from sensors 201, 213 and use that information to develop the virtual image of the probe on the sonogram.

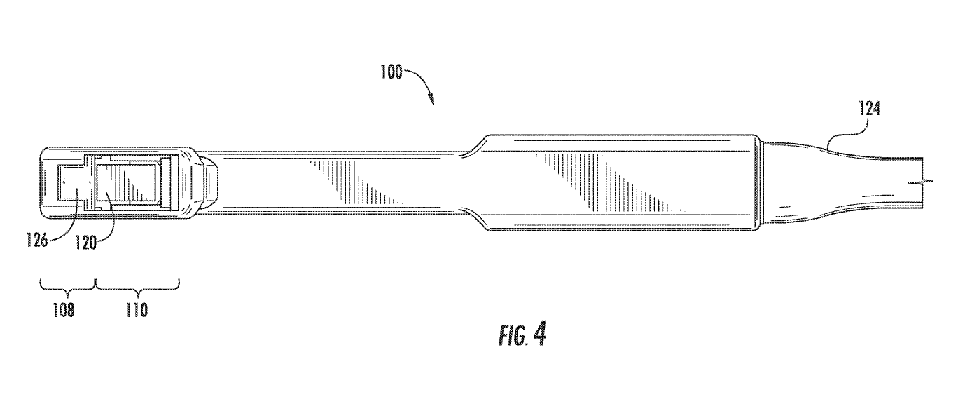

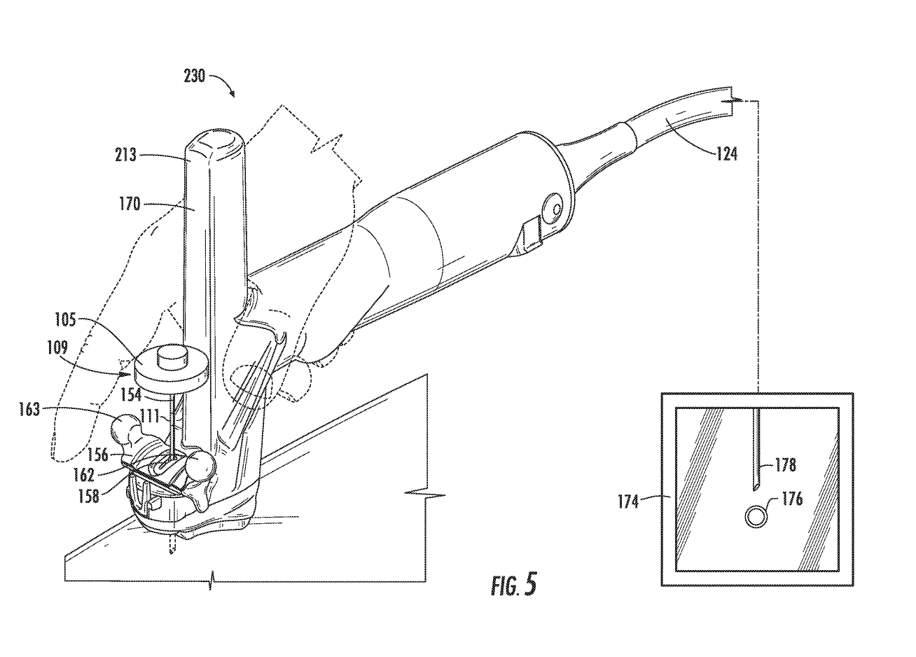

FIG. 3 illustrates one embodiment of an ultrasound transducer housing generally 100. Transducer housing 100 includes handle 102, post 104, and base 106. FIG. 4 provides a bottom view of transducer housing 100. An ultrasound transducer 120 that transmits and receives ultrasonic waves can be located in base 106, as shown in FIG. 4. Ultrasound transducer housing 100 can be formed of any suitable materials. For instance, any moldable polymeric material that can secure the ultrasound transducer 120 as well as contain associated electronics, wiring, switches, and the like and will not interfere with the functioning of the transducer 120 can be utilized.

Any type of ultrasound transducer as is generally known in the art can be incorporated in transducer housing 100. By way of example, a piezoelectric transducer formed of one or more piezoelectric crystalline materials arranged in a one or two-dimensional array can be utilized. For instance, a one dimensional array including a series of elements in a line can be used to create a two-dimensional image. Alternatively, a single transmitter can be moved through space to create two-dimensional image. A two-dimensional array can include a matrix of elements in a plane and can be used to create a three-dimensional image. A three-dimensional image can also be made by moving a two-dimensional array through space (rotationally or otherwise).

Transducer materials generally include ferroelectric piezoceramic crystalline materials such as lead zirconate titanate (PZT), although other suitable materials are encompassed herein, such as CMUT/PMUT materials.

An ultrasound transducer 120 can be formed of multiple elements. However, single transmitter/receiver devices are also encompassed by the present disclosure. The use of a multiple element ultrasound transducer can be advantageous in certain embodiments, as the individual elements that make up the array can be individually controlled. Such control systems are generally known in the art and thus will not be described in detail.

Ultrasound transducer housing 100 defines a probe guide opening 126 that passes through base 106. As can be seen in FIG. 4, probe guide opening 126 can be aligned with transducer 120. A probe that is guided through the probe guide opening 126 can travel on a path that is generally parallel to the scanned plane of a sonogram formed by use of the ultrasound device. In general, the scanned plane (i.e., the plane of the sonogram) is the geometric central plane of the beam transmitted from the ultrasound transducer 120. In one embodiment, the path of a probe guided through probe guide opening 126 can be within the scanned plane. This is not a requirement of the present disclosure, however. For instance, the path of a probe passing through probe guide can be at an angle to the scanned plane such that it intersects the scanned plane. By way of example, the line defined by the path of a probe passing through the probe guide can be at an angle of ±1° of the scanned plane in one embodiment, at an angle of ±0.6 degrees in another embodiment, or at a lesser or greater angle in another embodiment. For instance, a line defined by the path of a probe passing through the probe guide can be at an angle of ±10,°±20°, ±45°, or even greater, in other embodiments.

Ultrasound transducer 120 can be connected via signal wires in a cable 124 that leads to a processor that processes the data to form a sonogram on a monitor, as is generally known in the art. In the particular embodiment as illustrated in FIG. 3, a portion of cable 124 is internal to handle 102 of the ultrasound transducer housing 100, though this particular arrangement is not a requirement of the disclosure. Handle 102 can generally be set at an angle to post 104 of transducer housing 100 so as to be comfortably held in the hand while the device is being utilized. For instance, in the illustrated embodiment, handle 102 is about 90° to post 104, though this angle can be varied as desired. It should be understood however that a device need not include an extending handle portion at all.

As shown on FIGS. 3 and 4, base 106 defines a lower surface 108 defining probe guide opening 126 and lower surface 110 including transducer 120. Surfaces 108 and 110 together can form a skin contacting surface on the base 106 of the device 100. As can be seen in FIG. 3, surfaces 108 and 110 are contiguous and angled with respect to one another. The angle between surface 108 and 110 can vary, for instance in one embodiment the angle marked as θ in FIG. 3 can vary from 0 to about 30° or from about 10° to about 20° in another embodiment. Accordingly, the angle between surfaces 108 and 110 can be greater than about 150° and less than 180° in one embodiment, or greater than about 160° and less than about 170° in another embodiment.

There is no particular geometric configuration for transducer housing 100 and its individual sections that is essential to the system. For example, the base 106 of transducer housing 100 may be oblong, square, round, rectangular or any other suitable shape. In certain embodiments, the shape of transducer housing 100 may be particularly designed to fit specific locations of the anatomy. For example, transducer housing 100 may be shaped to be utilized specifically for infraclavicular approach to the subclavian vein, approach to the internal jugular vein, specific biopsy procedures including, without limitation, breast biopsy, thyroid nodule biopsy, prostate biopsy, lymph node biopsy, and so forth, or some other specific use. Variations in shape for any particular application can include, for example, a specific geometry for the footprint of base 106, alteration in the size of post 104 and/or handle 102, as well as variation in angles at which various elements of a device meet each other, such as the angle defined by the bottom of base 106 previously discussed. For example, the footprint of base 106 can be any suitable shape and size, e.g., rectangular, round, oblong, triangular, etc. By way of example, the skin contacting surface of base 106 can be between about 0.5 inches and about 6 inches on its greatest length. In one embodiment, the footprint of base 106 can be about 0.5 inches on its greatest width and can promote stability of the device during use. In other embodiments, it can be smaller or larger, however, such as about 1 inch on its greatest width, about 2 inches on its greatest width, or even larger.

Transducer housing 100 can be used as is, with no additional shield or covering over the housing 100. According to this embodiment, a probe, e.g., a needle, can pass through probe guide opening 126 and can be directed to a target that is visualized on a sonogram formed by use of ultrasound transducer 120.

An ultrasound device can include an ultrasound transducer housing that can be utilized in conjunction with a sterilizable shield, for instance in those embodiments in which a probe is intended for use in a sterile field. According to this embodiment, a transducer housing can be utilized in conjunction with a sterilizable shield that can provide a sterile barrier between a patient and all or a portion of the ultrasound transducer housing during a medical procedure.

A sterilizable shield can be formed of sterilizable materials as are generally known in the art. In one embodiment, a sterilizable shield can be formed of single-use materials such as polymeric materials and the entire shield can be properly disposed of following a single use. In another embodiment, a sterilizable shield can be utilized multiple times, in which case it can be formed of a material that can be properly sterilized between uses. By way of example, a sterilizable shield can be formed of a moldable thermoplastic or thermoset polymeric material including, without limitation, polyester, polyvinyl chloride, polycarbonate, and so forth. A sterilizable shield may also be formed of pliable materials, such as pliable films or sheets that can wrap around all or a portion of an ultrasound device. Combinations of materials may also be utilized, such as a molded plastic base attached to a pliable sheet that can fold over and wrap a portion of the ultrasound device.

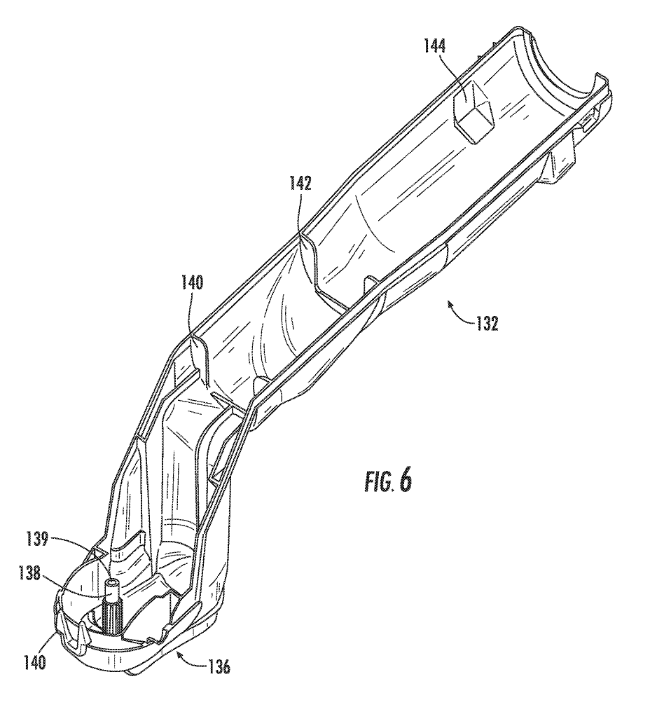

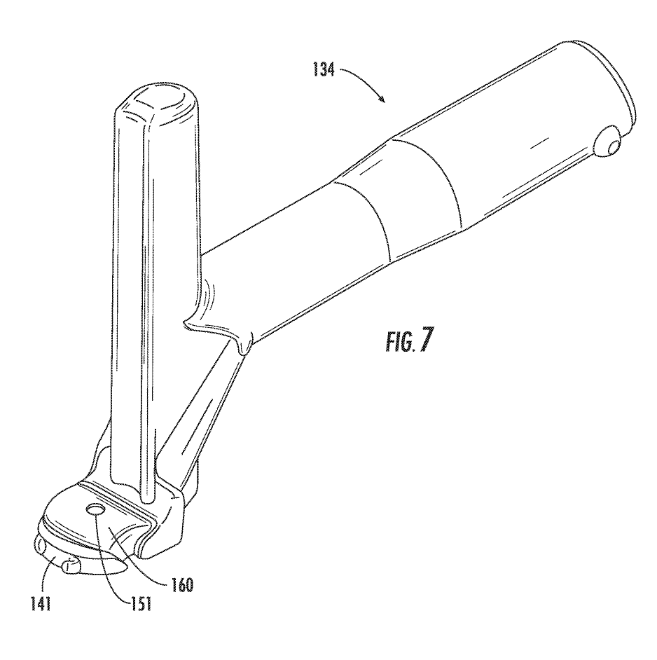

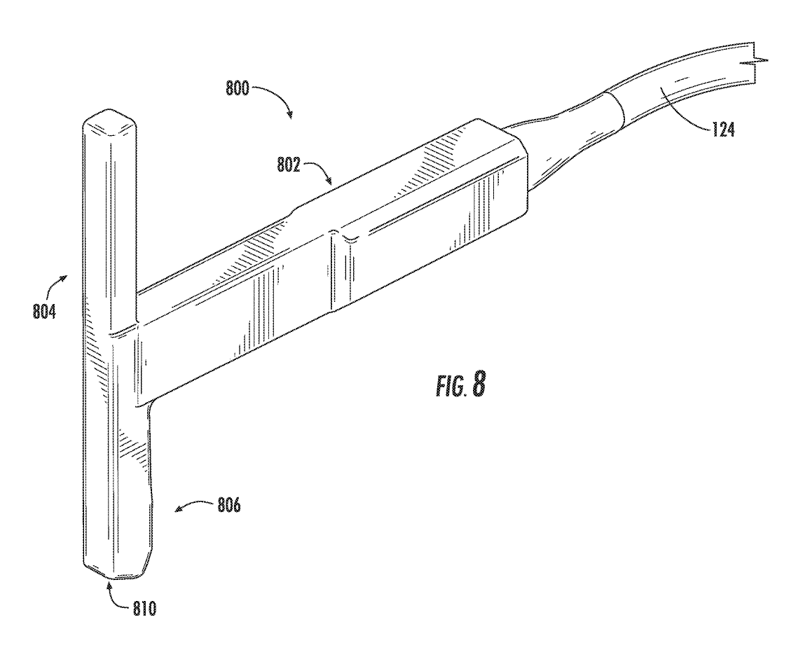

FIG. 5 illustrates one example of an ultrasound device encased in a sterilizable shield 230 during use. Sterilizable shield 230 can include a lower section 1132, details of which are shown in FIG. 6, and an upper section 134, details of which are shown in FIG. 7.

With reference to FIG. 6, shield lower section 1132 include a base 136 formed of an ultrasonic transmissive material. Base 136 can be of any suitable size and shape, but formed such that the ultrasound transducer housing base may be seated firmly in shield base 136. Generally, a small amount of an ultrasonic gel can be placed between the bottom surface of the transducer housing base and shield base 136 during seating to prevent any air between the two and promote transmission of ultrasonic waves.

Arising out of shield base 136 is guide post 138. Guide post 138 defines at least a portion of a probe guide 139 therethrough. Probe guide 139 extends uninterrupted completely through both guide post 138 and shield base 136. Guide post 138 can include tabs as shown, or other formations such as hooks, insets, or the like that can be utilized to properly assemble shield base 136 about ultrasound transducer housing 100. In one embodiment, guide post 138 may include a removable cap (not shown) for protection of the interior sterile surface of probe guide 139 during assembly of shield 230 with an ultrasound transducer housing.

As can be seen, shield lower section 1132 can also include tabs 1140, 140, 142, 144, etc. that can be utilized in properly seating a transducer housing within shield base 136 as well as aligning shield lower section 1132 with shield upper section 134 when assembling the complete shield 230 about an ultrasound transducer housing.

In the illustrated embodiment, tabs 140 on shield lower section 1132 match with corresponding notch 141 on shield upper section 134 shown in FIG. 7. Together tabs 140 and notch 141 form a fastener that can secure shield lower section 1132 and shield upper section 134 to one another. During assembly, tabs 140 can snap into notch 141 to securely fasten the two sections together and prevent separation of the sections 1132, 134 during use. Of course, a shield can include additional fasteners at other locations between the two sections, or can include a single fastener at an alternative location, as would be known to one of skill in the art.

Upper section 134 is illustrated in more detail in FIG. 7. Upper section 134 defines the terminal portion 151 of probe guide 139 shown in FIG. 6. Terminal portion 151 is sized so as to reside over the top of guide post 138 of lower section 1132 and form uninterrupted probe guide 139 extending from the top surface of portion 160 of upper section 134 to the bottom surface of base 136 of lower section 1132.

To assemble the illustrated sterilizable ultrasound device, ultrasound transducer housing 100 defining probe guide opening 126 shown in FIG. 3 can be seated in shield base 136 of lower section 1132 such that guide post 138 extends through transducer housing probe guide opening 126. As probe guide opening 126 of transducer housing 100 is slid over guide post 138, tabs on guide post 138 can slide or snap into recesses of probe guide opening 126 (not shown), helping to properly seat transducer housing 100 in lower section 1132. After ultrasound transducer housing 100 is seated in lower section 1132, upper section 134 can be aligned with lower section 1132 and fastened into place to cover the top of transducer housing 100. If a protective cap covers the end of guide post 138, it can be removed during assembly and maintain the sterility of the interior of the probe guide 139 throughout the assembly process. Tabs 140 can snap or slide into recesses notch 141 to fasten and secure lower section 1132 and upper section 134 together.

Following the above described assembly process probe guide 139 can extend continuously from the top of portion 160 of shield upper section 134 through the shield base 136. Moreover, and of great benefit to the device, probe guide 139 can be sterile and within the probe guide opening 126 of ultrasound transducer housing 100.

Though illustrated as being formed of two separable sections, a sterilizable shield can be hinged or can include additional sections, as desired. For instance, a sterilizable shield can be formed of two, three, or more separable sections that can be independently rigid, semi-rigid, or flexible. The sections can be assembled to enclose a transducer housing and form a sterile barrier between the enclosed housing and an exterior field. In another embodiment, a sterilizable shield can be of a unitary construction. For instance, a sterilizable shield can be of a pliant material that can enclose all or a portion of a transducer housing and form a sterile barrier between the enclosed housing and an exterior field.

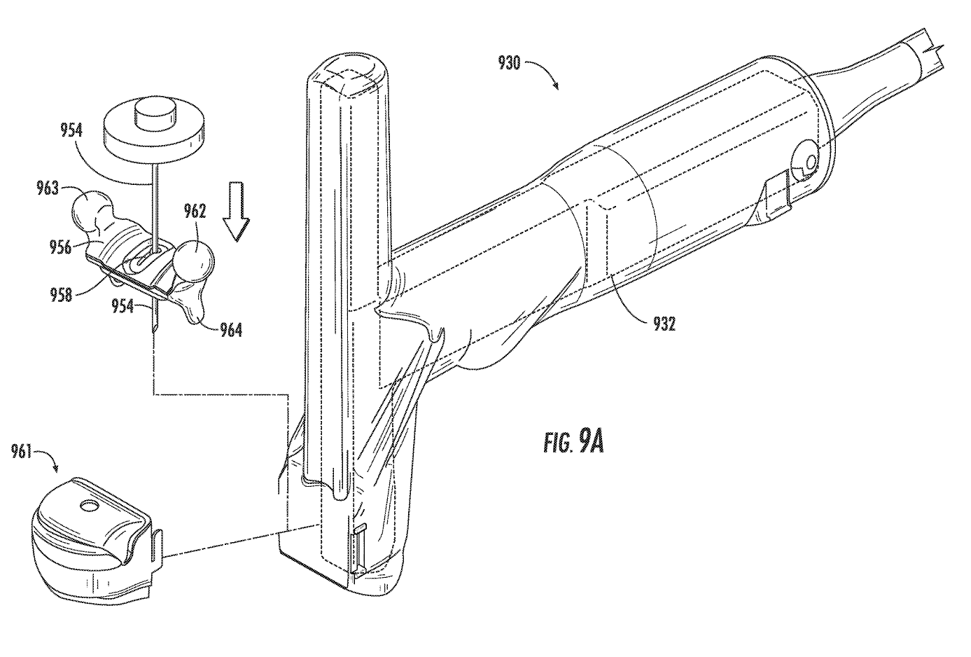

FIG. 8 illustrates another embodiment of an ultrasound transducer housing 800 that can be removably attachable to a sterilizable shield. According to this embodiment, ultrasound transducer housing 800 can include a handle 802, a post 804, and a base 806. Ultrasound transducer housing 800 also defines a lower surface 810, as shown. In this particular embodiment, however, the ultrasound transducer housing does not include a probe guide opening. Instead, ultrasound transducer housing 800 is removably attachable to a second portion of a device that defines the probe guide opening. For instance, ultrasound transducer housing 800 can be utilized in conjunction with a sterilizable shield that defines the probe guide. Moreover, the sterilizable shield can be formed of a single or multiple removably attachable pieces.

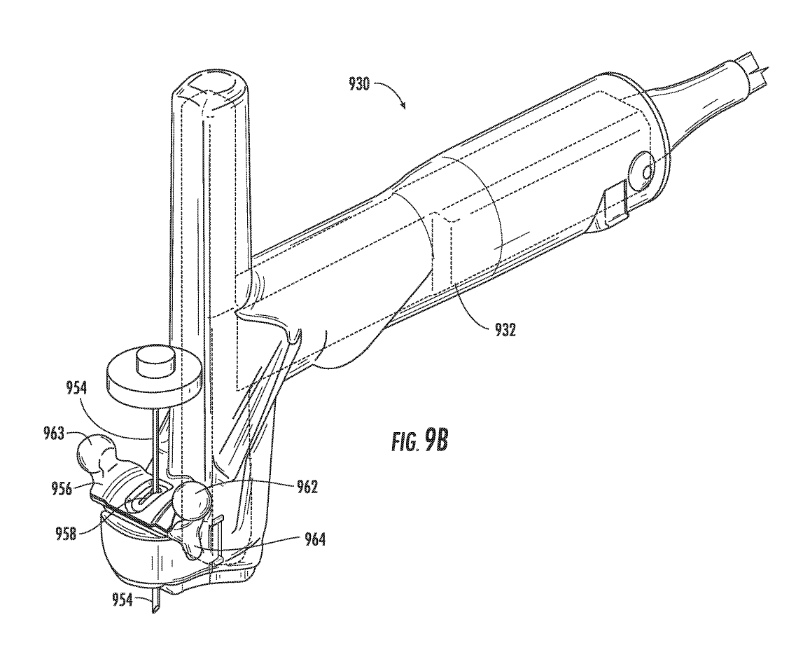

FIG. 9A and FIG. 9B illustrate one embodiment of a sterilizable shield 930 that can be used in conjunction with an ultrasound transducer housing 800 illustrated in FIG. 8. With reference to the exploded illustration of FIG. 9A, sterilizable shield 930 can enclose an ultrasound transducer housing 800. Sterilizable shield 930 can be formed of multiple attachable pieces. Specifically, sterilizable shield 930 includes section 932 and section 961 that defines a probe guide for passage of probe 954 therethrough. Additionally, section 932 can be separable into two or more section, as illustrated for the sterilizable shield of FIG. 6 and FIG. 7. Section 961 can also include clamp 956 defining aperture 958 and formations 962, 963 that rotates about pivot 964 for clamping probe 954 in the probe guide. During use, section 961 can be attached to shield 932, for instance by use of aligned tabs and notches, and so forth, so as to attach the probe guide portion to the sterilizable shield, as shown in FIG. 9B.

Of course, any other arrangement of the individual portions of a device is encompassed within the present disclosure. For instance, in one embodiment, an ultrasound transducer housing that does not define a probe guide opening, as illustrated in FIG. 8, can be removably attached to a piece that can define a probe guide opening, without enclosing all or a portion of the ultrasound transducer housing in a shield. In another embodiment, a sterilizable shield portion can cover only the skin contacting surface of a device. For instance, a shield portion can snap onto the base of a device.

By use of the probe detection system, the motion of the probe can be detected as can the characteristics of the probe and an image of a virtual probe can be added to the sonogram. More specifically, the probe detection system can include the motion detector and associated target that can register motion of a probe in the probe guide and can also include the information tag of the probe assembly that can provide information about the probe itself. The information from the probe detection system can be displayed as a real time virtual image of the probe on a sonogram. Thus, the location of the probe tip in relation to the target and the moment when the probe tip strikes the target can be seen in real time by an operator watching the virtual probe on the monitor during the procedure.

FIG. 5 illustrates the use of a system including an image of a virtual probe overlaid on a sonogram. In this particular embodiment, the probe device can include a detector 170 and a tag sensor 213 located in the post of the sterilizable shield 230 or in the post of the transducer housing enclosed within the shield 230. Detector 170 can recognize and monitor the movement of probe 154 as it passes through the probe guide and into a subject. Sensor 213 can obtain the identification information contained in tag 111. Information from detector 170, the sensor 213, and the ultrasound transducer can pass through cable 124 to a processor (not shown) and to a monitor 174. The probe 154 can then be imaged on a monitor 174 as virtual probe image 178. The monitor 174 can also show the internal target, for instance a blood vessel 176 on a sonogram.

Signals from detector 170 and sensor 213 can create a data stream which can be sent to a processor. A processor can be internal or external to the hand-held device. For example, data from detector 170 and sensor 213 can be sent to a standard laptop or desktop computer processor or part of a self-contained ultrasound system as is known in the art. A processor can be loaded with suitable recognition and analysis software and can receive and analyze the stream of data from detector 170 and sensor 213. The processor can also include standard imaging software as is generally known in the art to receive data from the ultrasound transducer via cable 124. Thus, through analysis of the data stream received from detector 170, from sensor 213, and from ultrasound transducer 120 (FIG. 4), a processor can be programmed to calculate the relative position of the probe tip in relation to the ultrasound transducer 120, in relation to detector 170, in relation to the exit of the probe guide, or to any other convenient reference point. A processor can communicate this position information digitally to monitor 174 and the information can be displayed on the monitor such as in a numerical format or optionally as a real time image of a virtual probe 178 shown in conjunction with the sonogram including an image of the target, such as a blood vessel 176.

In such a manner, disclosed devices can be utilized to show the approach of the probe toward the target on the monitor throughout the entire procedure, as the virtual probe location is highly coordinated with the actual probe location. In addition disclosed devices can be utilized to ensure the probe tip remains at the target during subsequent procedures. For example, in those embodiments wherein a detector 170 monitors the motion of the probe 154, as long as the detector is interacting with the probe, e.g., the sending and receiving of signals between the two, the image 178 of probe 154 can remain on the monitor 174. Thus, any motion of the probe tip in relation to the target can be noted by an observer, even following the clamping of the probe 154 within the probe guide by use of clamp 156.

Presently disclosed probe devices and methods may be utilized in many different medical procedures. Exemplary applications for the devices can include, without limitation

Central Venous Catheterization

Cardiac Catheterization (Central Arterial Access)

Dialysis Catheter Placement

Breast Biopsies

Paracentesis

Pericardiocentesis

Thoracentesis

Arthrocentesis

Lumbar Puncture

Epidural Catheter Placement

Peripherally Inserted Central Catheter (PICC) line placement

Thyroid Nodule Biopsies

Cholecystic Drain Placement

Amniocentesis

Regional Anesthesia—Nerve Block

Some of these exemplary procedures have employed the use of ultrasound in the past, and all of these procedures, as well as others not specifically listed, could utilize disclosed probe devices to improve procedural safety as well as patient safety and comfort, in addition to provide more economical use of ultrasound devices.

It will be appreciated that the foregoing examples, given for purposes of illustration, are not to be construed as limiting the scope of this invention. Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention which is defined in the following claims and all equivalents thereto. Further, it is recognized that many embodiments may be conceived that do not achieve all of the advantages of some embodiments, yet the absence of a particular advantage shall not be construed to necessarily mean that such an embodiment is outside the scope of the present invention.

What is claimed is:

1. An ultrasound system comprising:
a housing, the housing containing an ultrasound transducer, a magnetic field detector, and a tag sensor;
a probe assembly including a probe that is configured for being guided to a subdermal location, a tag that is detectable by the tag sensor, the tag carrying information concerning the geometry of the probe, and a target that is detectable by the magnetic field detector, the target consisting of one or more magnets;
a probe guide that is attachable to the housing, the probe guide defining an opening through which the probe is capable of passing, wherein upon attachment of the probe guide to the housing, the probe guide defines a barrier between the probe in the probe guide and the housing, the barrier precluding contact between the probe in the probe guide and the housing;
a monitor; and
a processor in communication with the detector, the tag sensor, the monitor, and the ultrasound transducer, the processor being configured to analyze data from the detector and the tag sensor to calculate a position of the probe in relation to a reference point, the processor being configured to communicate the position to the monitor, the monitor being configured to thereby display a real time image of the position as a virtual probe.

2. The ultrasound system of claim 1, wherein the magnetic field detector is integral to or removably attachable to the housing.

3. The ultrasound system of claim 1, wherein the magnetic field detector includes a series of sensors.

4. The ultrasound system of claim 3, wherein the sensors include Hall effect sensors.

5. The ultrasound system of claim 1, wherein the tag sensor is integral to or removably attachable to the housing.

6. The ultrasound system of claim 1, wherein the tag is a radio frequency identification tag.

7. The ultrasound system of claim 6, wherein the radio frequency identification tag is an active tag.

8. The ultrasound system of claim 6, wherein the radio frequency identification tag is a passive tag.

9. The ultrasound system of claim 1, wherein the tag is an optical tag.

10. The ultrasound system of claim 1, wherein the tag is a component of the target.

11. The ultrasound system of claim 1, wherein the probe includes a needle.

12. The ultrasound system of claim 11, the probe assembly further including a syringe, the tag being located on the needle, on the syringe hub, or on a support for the target.

13. The ultrasound system of claim 1, further comprising a clamp.

14. An ultrasound system comprising:
a probe assembly including a probe that is configured for being guided to a subdermal location, a tag that is detectable by a tag sensor, the tag carrying information concerning the geometry of the probe, and a target that is detectable by a magnetic field detector, the target consisting of one or more magnets; and
a sterilizable shield, the sterilizable shield being configured for attachment to an ultrasound transducer housing, the sterilizable shield including a probe guide, the probe guide defining an opening through which the probe is capable of passing, the probe guide being configured to preclude contact between the probe when held in the probe guide and an ultrasound transducer housing.

15. The ultrasound system of claim 14, the sterilizable shield including multiple non-pliable sections that are removably attachable to one another, wherein the probe guide is removably attachable to one of the non-pliable sections.

16. The ultrasound system of claim 14, wherein the probe guide includes a first portion and a second portion that are removably attachable to one another.

17. The ultrasound system of claim 14, wherein the tag is a radio frequency identification tag.

18. The ultrasound system of claim 17, wherein the radio frequency identification tag is an active tag.

19. The ultrasound system of claim 17, wherein the radio frequency identification tag is a passive tag.

20. The ultrasound system of claim 14, wherein the tag is an optical tag.

21. The ultrasound system of claim 14, wherein the tag is a component of the target.

22. The ultrasound system of claim 14, wherein the probe includes a needle.

23. The ultrasound system of claim 22, the probe assembly further including a syringe, the tag being located on the needle, on the syringe, or on a support for the target.

24. The ultrasound system of claim 14, further comprising a clamp.
